(19) United States
(12) Patent Application Publication
(10) Pub. No.: US 2023/0368863 A1
(43) Pub. Date: Nov. 16, 2023

(54) Multiplexed Screening Analysis of Peptides for Target Binding

(71) Applicant: Genentech, Inc., South San Francisco, CA (US)

(72) Inventors: Man-Ling Lee, San Francisco, CA (US); Alberto Emilio Gobbi, San Francisco, CA (US); Christian Nathaniel Cunningham, Oakland, CA (US)

(21) Appl. No.: 18/338,772

(22) Filed: Jun. 21, 2023

Related U.S. Application Data
(63) Continuation of application No. PCT/US2021/062258, filed on Dec. 7, 2021.
(60) Provisional application No. 63/129,077, filed on Dec. 22, 2020.

(51) Int. Cl. G16B 15/30; G16B 15/20; G16B 40/00

(52) U.S. Cl. CPC G16B 15/30; G16B 15/20; G16B 40/00

(57) ABSTRACT

Methods, systems, and computer program products are provided for clustering of similar peptides to detect candidates for target binding. In some embodiments, a method provided herein includes receiving sequencing information and quantification information of a plurality of peptides after target-binding selection in a library. The sequencing information includes amino acid sequences of the plurality of peptides, and the quantification information includes a count of copies of each amino acid sequence in the plurality of peptides. The method further includes computing similarity scores for pairs of the plurality of peptides using the sequencing information. The method further includes grouping the plurality of peptides into clusters based on the similarity scores. The method further includes screening the clusters based on quantification information of peptides in each cluster to obtain candidates for target binding over a pre-set threshold.

### PRIORITY

This application claims the benefit under 35 U.S.C. § 365(c) of International Patent Application No. PCT/US2021/062258, filed 7 Dec. 2021, which claims the benefit under 35 U.S.C. § 119(e) of U.S. Provisional Patent Application No. 63/129,077, filed 22 Dec. 2020, each of which is incorporated herein by reference in its entirety.

### TECHNICAL FIELD

Provided herein are methods and systems for improved multiplexed screening analysis. More specifically, methods and systems are provided for multiplexed screening of nucleotide-tagged peptide libraries for target-binding activity by clustering of peptides based on similarity.

### BACKGROUND

Current multiplexed target-binding candidate screening analysis systems have difficulty with the selection of many nucleotide-containing peptide libraries for binding to a desired target due to problems such as low sensitivity and false negatives. That is, conventional screening analysis systems are ineffective at detecting similar peptides which individually would show insufficient target binding activity. There is, therefore, a need for improved multiplexed target-binding candidate screening analysis systems and methods to help selection of candidate binders against a desired binding target, e.g., a protein.

### SUMMARY OF PARTICULAR EMBODIMENTS

The embodiments described herein provide various methods, systems, and computer program products for clustering of similar peptides to detect candidates for target binding.

In some embodiments, a method is provided for detecting candidates for target binding. The method includes receiving sequencing information and quantification information of a plurality of peptides after target-binding selection in a library. The sequencing information includes amino acid sequences of the plurality of peptides, and the quantification information includes a count of copies of each amino acid sequence in the plurality of peptides. The method further includes computing similarity scores for pairs of the plurality of peptides using the sequencing information. The method further includes grouping the plurality of peptides into clusters based on the similarity scores. The method further includes screening the clusters based on quantification information of peptides in each cluster to obtain candidates for target binding over a pre-set threshold.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the claimed embodiments. Thus, it should be understood that although the present claimed embodiments have been specifically disclosed as embodiments and optional features, modification and variation of the concepts herein disclosed may be resorted to by those skilled in the art, and that such modifications and variations are considered to be within the scope of the appended claims.

### BRIEF DESCRIPTION OF THE DRAWINGS

The present disclosure is described in conjunction with the appended figures:

FIG. 1 illustrates non-limiting exemplary embodiments of a general schematic workflow for screening a plurality of libraries for binding to a desired binding target, in accordance with various embodiments.

FIG. 2 illustrates non-limiting exemplary embodiments of a general schematic workflow for clustering of peptides to detect candidates for target binding, in accordance with various embodiments.

FIG. 3 illustrates non-limiting exemplary embodiments of an amino acid similarity matrix, in accordance with various embodiments.

FIG. 4 illustrates non-limiting exemplary embodiments of a distribution of similarity scores, in accordance with various embodiments.

FIG. 5 illustrates non-limiting exemplary embodiments of a graph showing frequency of all peptides in each cluster, in accordance with various embodiments.

FIG. 6 illustrates non-limiting exemplary embodiments of a graph showing similarity scores of all peptides in each cluster, in accordance with various embodiments.

FIG. 7 illustrates non-limiting exemplary embodiments of a graph showing a sum of frequencies of all peptides in each cluster verse a size of each cluster, in accordance with various embodiments.

FIG. 8 is a flowchart illustrating a method for clustering of peptides to detect candidates for target binding, in accordance with various embodiments.

FIG. 9 is a flowchart illustrating a method for clustering of peptides to detect candidates for target binding, in accordance with various embodiments.

FIG. 10 illustrates non-limiting exemplary embodiments of a system for clustering of peptides to detect candidates for target binding, in accordance with various embodiments.

FIG. 11 is a block diagram of non-limiting examples illustrating a computer system configure to perform methods provided herein, in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DESCRIPTION OF EXAMPLE EMBODIMENTS

### I. Overview

Conventional screening analysis systems are ineffective at detecting similar peptides which individually would show insufficient target binding activity. However as described herein, similar peptides collectively (as a class or cluster of peptides) may indicate target binding activity that warrants further investigation, even though the individual peptides of that cluster would show insufficient target binding activity on their own.

This disclosure describes various exemplary embodiments for improved multiplexed target-binding candidate screening analysis systems and methods to help selection of candidate binders against a desired binding target, e.g., a protein. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

### II. Exemplary Context and Descriptions of Terms

It is to be understood that the terminology used herein is for the purpose of describing particular embodiments only, and is not intended to be limiting.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Generally, nomenclatures utilized in connection with, and techniques of, chemistry, biochemistry, molecular biology, pharmacology and toxicology are described herein are those well-known and commonly used in the art.

As used herein, the singular forms “a” “an” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes” “including” “comprises” and/or “comprising” when used herein, specify the presence of stated features, integers, steps, operations, elements, components, and/or units but do not preclude the presence or addition of one or more other features, integers, steps, operations, elements, components, units, and/or groups thereof.

Throughout this disclosure, various aspects are presented in a range format. It should be understood that the description in range format is merely for convenience and brevity and should not be construed as an inflexible limitation on the disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed in the disclosure. The upper and lower limits of these smaller ranges may independently be included in the smaller ranges, and are also encompassed in the disclosure, subject to any specifically excluded limit in the stated range. Where the stated range includes one or both of the limits, ranges excluding either or both of those included limits are also included in the disclosure. This applies regardless of the breadth of the range.

The term “about” as used herein refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, ±5%, or ±1% as understood by a person of skill in the art.

In addition, as the terms “in communication with” or “communicatively coupled with” or similar words are used herein, one element may be capable of communicating directly, indirectly, or both with another element via one or more wired communications links, one or more wireless communications links, one or more optical communications links, or a combination thereof. In addition, where reference is made to a list of elements (e.g., elements a, b, c), such reference is intended to include any one of the listed elements by itself, any combination of less than all of the listed elements, and/or a combination of all of the listed elements.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “ones” means more than one.

As used herein, the term “plurality” or “group” can be 2, 3, 4, 5, 6, 7, 8, 9, 10, or more.

As used herein, the term “set” means one or more.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” or “at least one of item A, item B, and item C” may mean item A; item A and item B; item B; item A, item B, and item C; item B and item C; or item A and C. In some cases, “at least one of item A, item B, or item C” or “at least one of item A, item B, and item C” may mean, but is not limited to, two of item A, one of item B, and ten of item C; four of item B and seven of item C; or some other suitable combination.

An “individual”, “subject,” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “nucleotide,” “polynucleotide,” “nucleic acid,” or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

As used herein, a “genome” is the genetic material of a cell or organism, including animals, such as mammals, e.g., humans. In humans, the genome includes the total DNA, such as, for example, genes, noncoding DNA and mitochondrial DNA. The human genome typically contains 23 pairs of linear chromosomes: 22 pairs of autosomal chromosomes plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

The phrase “sequencing” refers to any technique known in the art that allows the identification of consecutive nucleotides of at least part of a nucleic acid. Non-limiting exemplary sequencing techniques include RNA-seq (also known as whole transcriptome sequencing), Illumina™ sequencing, direct sequencing, random shotgun sequencing, Sanger dideoxy termination sequencing, whole-genome sequencing, massively parallel signature sequencing (MPSS), sequencing by hybridization, pyro sequencing, capillary electrophoresis, gel electrophoresis, duplex sequencing, cycle sequencing, single-base extension sequencing, solid-phase sequencing, high-throughput sequencing, massively parallel signature sequencing, emulsion PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, mass spectrometry, and any combination thereof.

The phrase “RNA-seq (RNA-sequencing)” refers to any step or technique that can examine the presence, quantity or sequences of RNA in a biological sample using sequencing such as next generation sequencing (NGS). RNA-seq can analyze the transcriptome of gene expression patterns encoded within the RNA.

The phrase “next generation sequencing” (NGS) refers to sequencing technologies having increased throughput as compared to traditional Sanger and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes.

The term “sequencing information” refers to nucleotide or amino acid sequences. In some embodiments, the sequencing information comprises amino acid sequences of a plurality of peptides.

The term “quantification information” refers to a count of copies of each peptide or nucleic acid sequence. In some embodiments, the quantification information includes a count of copies of each amino acid sequence in a plurality of peptides. Each amino acid sequence can represent a distinct peptide that is different from other peptides in at least one, two, three, four, five, six, seven, eight, nine, or more amino acid positions. A distinct peptide is different from other peptides in at least one, two, three, four, five, six, seven, eight, nine, or more amino acid positions. After undergoing target-binding selection of a library against a desired target, the library can contain one or more instances of each distinct peptide in a solution that were selected as initial candidates for binding to the desired target.

The term “clustering,” as used herein, refers to grouping a set of peptides in such a way that peptides in the same group (i.e., the same cluster) are more similar to each other than those in other groups (i.e., clusters).

The term “similarity matrix” as used herein refers to a matrix that measures similarities of any two amino acids, including natural and non-natural amino acids. The similarity matrix is different from an amino acid substitution scoring matrix, which measures the rates at which various amino acid residues in proteins are substituted by other amino acid residues, over time.

The term “selecting” used in a target-binding selection refers to substantially partitioning a molecule from other molecules in a population. As used herein, a “selecting” step provides at least a 2-fold, preferably a 30-fold, more preferably a 100-fold, and most preferably a 1000-fold enrichment of a desired molecule relative to undesired molecules in a population following the selection step. As indicated herein, a selection step may be repeated any number of times, and different types of selection steps may be combined in a given approach.

### III. Target-Binding Candidate Discovery

Various method and system embodiments described herein enable improved multiplexed methods to detect for peptide candidates in selection for binding to a desired target. For example, RNA display methods can be used here. RNA display generally involves expression of proteins or peptides, wherein the expressed proteins or peptides are linked covalently or by tight non-covalent interaction to their encoding mRNA to form RNA/protein fusion molecules. The protein or peptide component of an RNA/protein fusion can be selected for binding to a desired target and the identity of the protein or peptide determined by sequencing of the attached encoding mRNA component.

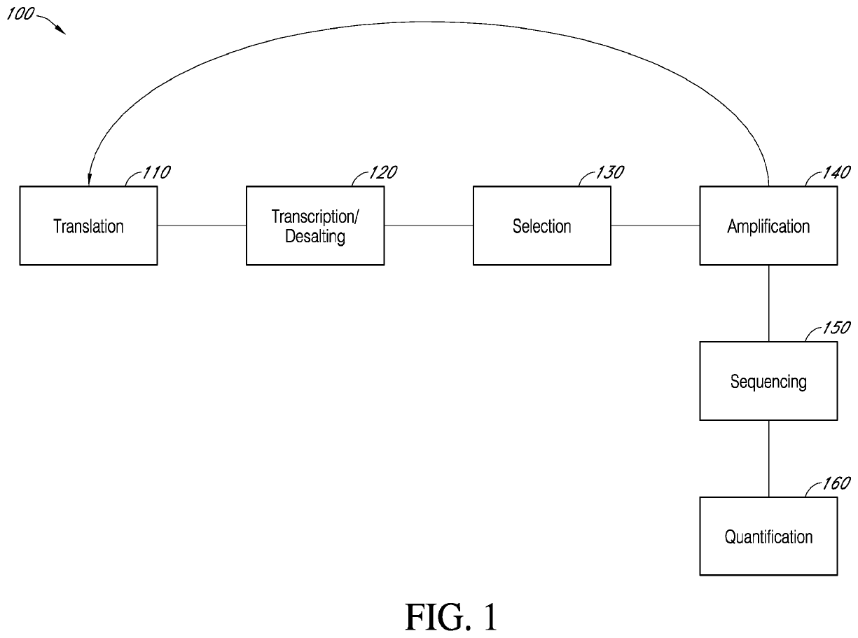

FIG. 1 illustrates non-limiting exemplary embodiments of a general schematic workflow for screening a plurality of libraries of DNA-containing compositions for binding to a desired target, in accordance with various embodiments.

The workflow 100 can include, at step 110, obtaining starting nucleic acid libraries (e.g., wells in a multi-well plate) and translating the starting nucleic acid libraries into peptide libraries that are encoded by their corresponding nucleic acids to produce libraries of nucleotide-containing conjugates. The starting nucleic acid libraries can include at least, at most, or about 10, 100, 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ (or any intermediate numbers of ranges derived therefrom) conjugates. The starting nucleic acid libraries can be chosen with a design preference. For example, the starting nucleic acid libraries can be chosen to have a low abundance of conjugates and can include about 10, 100, or 10³ (or any intermediate numbers of ranges derived therefrom) conjugates. The starting nucleic acid libraries can be chosen to have a medium abundance of conjugates and can include about 10⁴, 10⁵, 10⁶, 10⁷, 10⁸, or 10⁹ (or any intermediate numbers of ranges derived therefrom) conjugates. The starting nucleic acid libraries can be chosen to have a high abundance of conjugates and can include about 10¹⁰, 10¹¹, 10¹², 10¹³, or 10¹⁴ (or any intermediate numbers of ranges derived therefrom) conjugates.

The workflow 100 translates RNA to peptides by adding an in vitro translation mix, according to some embodiments. For example, the in vitro translation mix includes a ribozyme that charges tRNA with standard amino acids, a ribozyme that charges tRNA with non-standard amino acids, or a combination thereof, such as an aminoacyl-tRNA synthetase (aaRS or ARS or also called tRNA-ligase) for adding standard amino acids, a flexizyme for adding non-standard amino acids, or a combination thereof. During the in vitro translation reaction, the mRNA molecules become covalently linked to their peptide products via a peptide acceptor (e.g., puromycin) fused at the 3′ end. In additional and alternative embodiments, the nucleotide-containing conjugates may include linkers that link mRNA to the corresponding peptides.

The peptide can be linear, stapled, cyclic, or a combination thereof. In particular embodiments, the cyclic peptide is a macrocyclic peptide. The macrocyclic peptide can have one, two, three, or more rings. The macrocyclic peptide can include monocycle peptides, bicycle peptides or tetracycle peptides, or a combination thereof. The libraries of nucleotide-containing conjugates may include RNA conjugated to peptides as mRNA-displayed peptides.

The workflow 100 can include, at step 120, in vitro reverse transcription of nucleotide-containing conjugates and desalting the in vitro reverse transcription product. For example, the workflow 100 produces DNA-mRNA-peptide conjugates by adding a reverse transcription mix to mRNA-peptide conjugates. The workflow 100 transfers the resulting DNA-mRNA-peptide conjugates to desalting columns to remove salts and other small molecules, so desalted libraries are produced. The desalted libraries may be input for a round of selection to detect for target-binding candidate peptides.

The workflow 100 can include, at step 130, selection of target-binding candidates from input libraries. The input libraries may include the nucleotide-containing conjugates after in vitro reverse transcription and desalting. Each selection may include positive selection for candidate binders binding to a desired target molecule, negative selection to remove libraries that bind to support without the desired target molecule, or a combination thereof.

For example, the target molecules are bound to a solid support, such as agarose beads. The target molecule is directly linked to a solid substrate. In another embodiment, the target molecule is first modified, for example, biotinylated, then the modified target molecule is bound via the modification to a solid substrate, such as a bead. Non-limiting examples of a solid-support include streptavidin (SA)-M280, neutravidin-M280, SA-M270, NA-M270, SA-MyOne, NA-MyOne, SA-agarose, and NA-agarose. In additional and alternative embodiments, the solid support further includes magnetic beads, for example Dynabeads®. Such magnetic beads allow separation of the solid support, and any bound nucleotide-containing conjugates, from an assay mixture using a magnet.

In negative selection, the input libraries can be mixed thoroughly with empty beads. Any bead-binding members from the input libraries can be removed. In some embodiments, the first round of selection skips negative selection.

In positive selection, the input libraries can be incubated with one or more target molecules bound to a solid support, e.g., beads that capture tags displayed on one or more target molecules. For example, a pull-down assay can be performed to wash off unbound nucleotide-containing conjugates and elute candidate binders from beads that are attached to a target protein, i.e., positive beads.

The target-bound nucleotide-containing conjugates can be eluted from the solid support prior to amplification of the nucleic acid component. Any available method of elution is contemplated. Alternatively or additionally, the target-bound nucleotide-containing conjugates can be eluted at a high temperature, e.g., boiling. Alternatively or additionally, the target-bound nucleotide-containing conjugates are eluted using alkaline conditions, for example, using a pH of about 8.0, 8.5, 9.0, 9.5, 10.0, or any intermediate ranges or values derived therefrom. In additional and alternative embodiments, the target-bound nucleotide-containing conjugates are eluted using acid conditions, for example, using a pH of about 3.0, 3.5, 4.0, 4.5, 5.0, 5.5, 6.0, or any intermediate ranges or values derived therefrom.

For example, the positive beads can be transferred to a PCR plate, sealed, and boiled. The positive beads can then be cooled and transferred to a magnetic plate. The supernatant from the magnetic plate can be removed and transferred to a new PCR plate for further analysis of the nucleotide-containing conjugates.

The workflow 100 can include, at step 140, amplification of selected target-binding candidates from the input libraries. For example, selected target-binding candidates are DNA-RNA-peptide conjugates. The workflow 100 amplifies DNA in selected target-binding candidates by PCR and uses the amplified product as input for the next round of selection or analyzed by sequencing.

The workflow 100 further quantifies and normalizes, at step 140, selected target-binding candidates for DNA amplification in optional aspects. The workflow 100 measures DNA concentration in selected target-binding candidates, for example by quantitative PCR (qPCR). In optional aspects, the workflow 100 collects and analyzes qPCR data for normalization to ensure appropriate DNA concentration to be used in the next round of selection.

In additional and alternative embodiments, RNA in selected target-binding candidates may be amplified to produce more RNA. Any available method of RNA replication is contemplated, for example, using an RNA replicase enzyme. In another embodiment, RNA in eluted target-binding candidates may be transcribed into cDNA before being amplified by PCR.

In additional and alternative embodiments, the amplified nucleic acid sequences may be amplified under conditions that result in the introduction of mutations into amplified DNA, thereby introducing further diversity into the selected nucleic acid sequences. This mutated pool of DNA molecules may be subjected to further rounds of selection.

The workflow 100 can include, at steps 130 and 140, repeated selection of target-binding candidates from input libraries. The PCR-amplified pool can be subject to one or more rounds of selection to enrich for the highest affinity target-binding candidates, for example, two, three, four, five, six, seven, eight, nine, ten or more rounds. The process of selection and amplification is repeated until the libraries are dominated by candidates with the desired properties. The number of repetitions needed depends on the diversity of the starting libraries and the enrichment achieved in the selection step.

Amplified DNA nucleotides may be transcribed to mRNA and then translated to peptides to produce additional libraries of nucleotide-containing conjugates for another round of selection via steps 110, 120, 130, and 140.

At step 150, at the end of target-binding selection, the selected nucleic acids in selected nucleotide-containing conjugates may be sequenced using any available sequencing methods (e.g., next generation sequencing (NGS)) to determine the nucleic sequences of every selected nucleotide-containing conjugate. The sequence identity of selected nucleotide-containing conjugates can be further used for validation of target binding affinity of selected nucleotide sequences.

At step 160, the selected nucleic acids may be quantified using any available quantification methods (e.g., RT-PCR) to determine quantification information of every selected nucleotide-containing conjugate. The quantification information of every selected nucleotide-containing conjugate may include a count of copies of each amino acid sequence in a plurality of peptides, and the sequence identity of each amino acid sequence may be derived from sequencing of corresponding nucleotide sequences in each selected nucleotide-containing conjugate at step 150. Because the nucleic acids in each nucleotide-containing conjugate generate corresponding peptides in the same nucleotide-containing conjugate, the sequence identity and count of copies of the peptides can be derived from the corresponding nucleotide sequences.

### IV. Clustering of Peptides to Detect Candidates for Target Binding

Various method and system embodiments described herein enable improved screening of target-binding candidates, e.g., target-binding selection using in vitro display. In particular, the embodiments described herein enable identifying previously unidentified target-binding candidates using traditional methods. The methods and systems described herein are sensitive and reproducible and may be used to improve efficacy and yield of any screening analysis, particularly target-binding screening analysis.

IV.A. Clustering Workflow

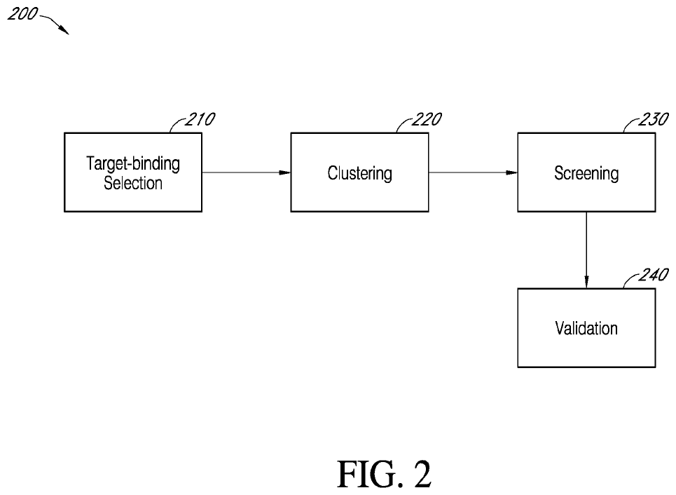

A general schematic workflow 200 is provided in FIG. 2 to illustrate a non-limiting example process for clustering of peptides to detect candidates for target binding in accordance with various embodiments. This allows for detection of peptides that may individually occur at low frequency, but when clustered into a group based on their relative similarity with each other that may instead (for some cluster in some instances) appear as high frequency in aggregate, thus suggesting that they are viable candidates for target binding.

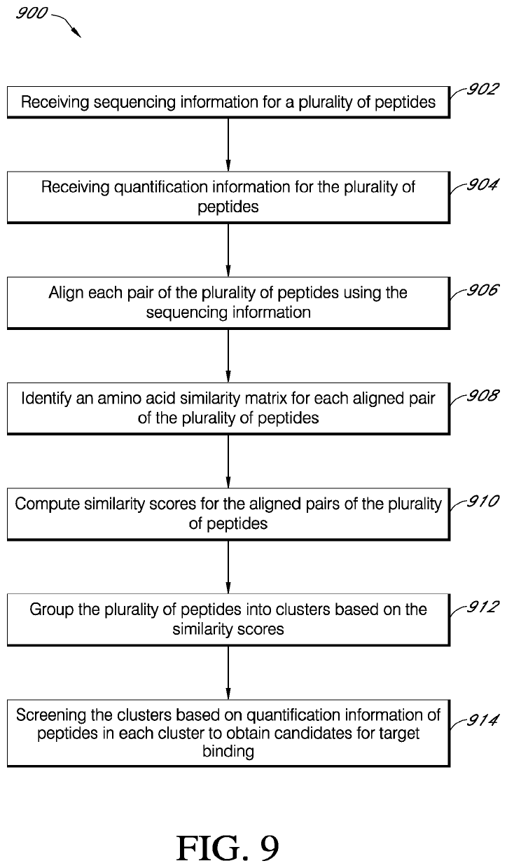

The workflow can include various combinations of features, whether it be more or less features than that illustrated in FIG. 2. As such, FIG. 2 simply illustrate one example of a possible workflow. The workflow 200 may be implemented using, for example, system 900 described with respect to FIG. 9 or a similar system.

The workflow 200 can include, at step 210, performing one or more rounds of selection to detect for binding to a desired target molecule. Each round of selection may start with translation, reverse transcription, desalting, selection to detect for binding to a target molecule, and quantification and sequencing of nucleotides from selected nucleotide-containing compositions to obtain sequencing information and quantification information of these selected nucleotide-containing compositions, as exemplified in FIG. 1. Amplification of nucleotides may be an optional step after target-binding selection (i.e., selection to detect for binding to a target molecule) to enrich candidates that may be of interest.

In particular aspects, the step 210 may include one or more of performing in vitro transcription of a DNA library to produce mRNA, performing in vitro translation on mRNA to produce RNA-peptide conjugates, performing in vitro reverse transcription on the RNA-peptide conjugates to produce input DNA-RNA-peptides as input libraries, incubating the input libraries with a desired target, such as a target protein, and selecting for target-binding candidates, such as target-binding DNA-RNA-peptides from the input libraries, wherein the target-binding candidates remain after the target-binding selection and are herein defined as the initial candidate peptides after the target-binding selection (and sometimes simply, “the peptides” or “the plurality of peptides” for brevity) for convenience of discussion below. As the name suggests, these initial candidate peptides are considered initial candidates for binding to the desired target. For example, the peptides may include DNA-RNA-peptides, such as DNA-RNA-macrocycle conjugates, wherein at least one of the peptides includes natural and non-natural amino acids. In various embodiments, the peptides are made using a codon table encoding natural amino acids, a codon table encoding non-natural amino acids, or a combination thereof.

The workflow 200 can include, at step 220, grouping peptides based on their similarity. For example, the workflow 200 may obtain or receive sequencing information and quantification information of the nucleotide-containing compositions after target-binding selection of a library of such nucleotide-containing compositions.

The nucleotide-containing compositions include a plurality of peptides, more particularly, peptide-nucleotide conjugates, such as DNA-RNA-macrocycle peptide conjugates. The sequencing information may include amino acid sequences of the plurality of peptides. In some aspects, the sequencing information of the plurality of peptides may be determined from corresponding DNA sequences in the conjugates, such as DNA-RNA-peptide conjugates, more particularly DNA-RNA-macrocycle conjugates. The workflow 200 may further comprise sequencing the DNA component in the selected DNA-RNA-peptides to determine the sequencing information for the plurality of peptides after target-binding selection.

The quantification information may include a count of copies of each instance of each distinct peptide in the plurality of peptides and can be used to determine a frequency of each distinct peptide in a cluster. Each amino acid sequence can represent a distinct peptide that is different from other peptides in at least one, two, three, four, five, six, seven, eight, nine, or more amino acid positions. In some embodiments, the quantification information of the plurality of peptides may be determined from counting DNA copies in the conjugates, such as DNA-RNA-peptide conjugates, more particularly DNA-RNA-macrocycle conjugates. The workflow 200 may further include amplifying the target-binding DNA-RNA-peptides by PCR to determine the quantifying information for the plurality of peptides after target-binding selection.

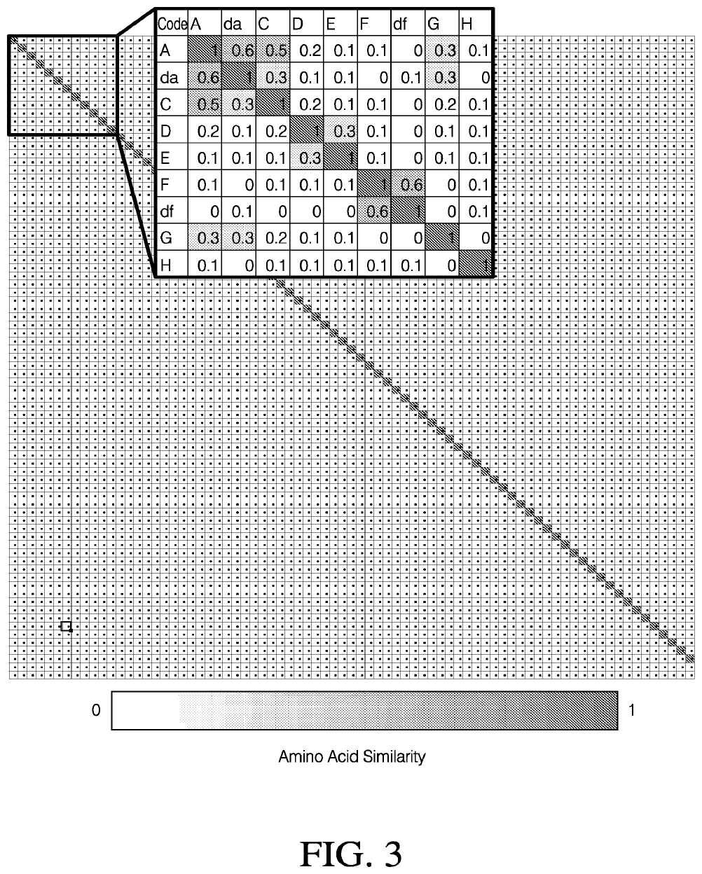

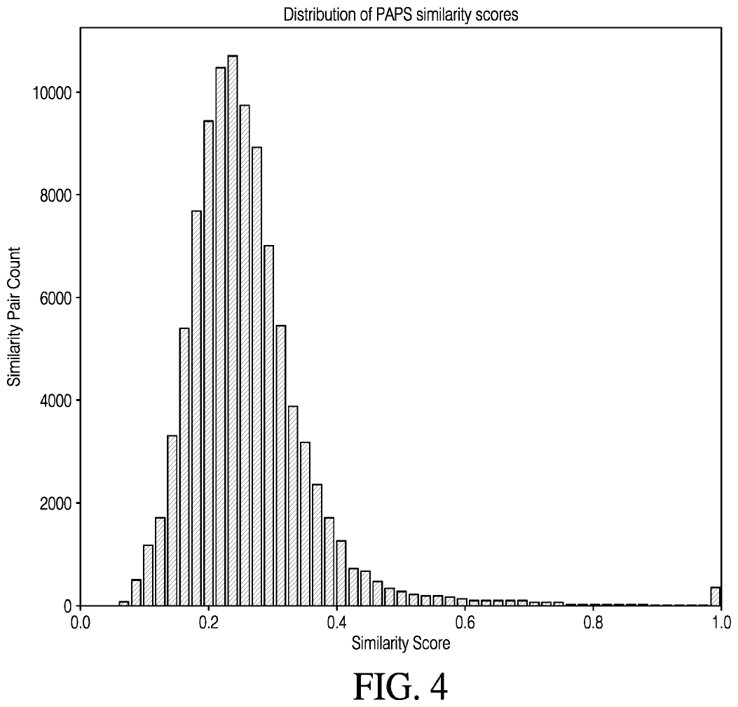

In various embodiments, the workflow 200 may compute similarity scores for the plurality of peptides using the sequencing information, e.g., similarity scores for pairs of the plurality of the peptides. The similarity score may be defined as pairwise aligned peptide (PAP) similarity in some embodiments. For example, the workflow 200 may include aligning each pair of the plurality of peptides using the sequencing information to generate a numerical measure of similarity for each pair of the plurality of peptides. Computing the similarity scores between any pair of peptides may include using a numerical measure of similarity based on an alignment between the peptides of each pair using an amino acid similarity matrix. An example of an amino acid similarity matrix is illustrated in FIG. 3. Further, a distribution plot of a pairwise aligned peptide (PAP) similarity score verse a similarity pair count from an exemplary library is illustrated in FIG. 4.

In an additional and alternate embodiment, a Round Robin variation may be used as a variation of the alignment algorithm described above. In this instance, the amino acids in the short sequence of each pair of sequences can shift a fixed number of positions in the same direction for an adjusted alignment and can be used to calculate a similarity score for the pairs of peptides using the adjusted alignment. For example, the Round Robin alignment is repeated with the amino acids of the second sequence shifting one position to the right and the amino acid on the far right shifting to the first position. This shifting is repeated until the amino acids return to their original position. The Round Robin variation increases the pool of alignments for each pair, from which the alignment with the highest alignment score can be picked as the optimal alignment for the given pair. In a particular example, sequence 1 and 2 of a pair can be aligned optimally without gaps.

The workflow 200 may further include obtaining a pre-determined amino acid similarity matrix that was previously generated. The workflow 200 may also include generating an amino acid similarity matrix, such as a chemical similarity matrix, for being used in the workflow 200, in some embodiments.

The chemical similarity matrix can consider the molecular structure similarities of amino acids pairs. By using this matrix, the similarity score can compare peptides comprising unnatural amino acids. In addition, the atom level description of the chemical similarity matrix in some aspects can be used for describing differences relevant for protein-ligand interactions.

For example, the chemical similarity matrix may be based on a stereochemistry-aware matrix that can distinguish amino acids based on alpha carbon (Cα) stereochemistry. The stereochemistry-aware matrix can distinguish two molecules such as, for example, two amino acids, that are otherwise identical but have different stereo-chemistries such as, for example, different relative spatial arrangement of atoms. For example, the amino acid similarity matrix may include a combination of a regular amino acid similarity matrix via a first pre-determined coefficient and a stereochemistry-aware amino acid similarity matrix via a second pre-determined coefficient.

Since the backbones of macrocycles are constrained, the stereochemistry at the α-carbon atoms are likely to have large impact on the binding to proteins. A stereochemistry-aware matrix, also referred to as a D/L isomer aware similarity matrix, is used to address the impact of such stereochemistry.

For a chemical similarity matrix, two initial similarity matrices can be generated in some examples: a first similarity matrix Sim_{i,j}^{no-stereo} can be generated using unmodified input amino acid structures; a second D/L isomer aware similarity matrix Sim_{i,j}^{no-stereo} can be generated using amino acid structures whose α-carbon atoms were replaced by Silicon (Si) in case of L-isomers or Germanium (Ge) atoms otherwise. The final chemical similarity matrix can be generated by combining the corresponding elements in Sim_{i,j}^{no-stereo} and Sim_{i,j}^{stereo} as described below in Equation 1. Accordingly, similarity scores for each amino acid pair, i and j, in two aligned peptides, can be generated as Sim_{i,j}.

Sim_{i,j}=c*Sim_{i,j}^{no-stereo}+(c−1)*Sim_{i,j}^{stereo} (Equation 1)

The weighing parameter c allows for tuning the impact of the stereochemistry on α-carbon atoms. The weighing parameter can be 0, 0.01, 0.1, 0.2, 0.3, 0.4, 0.5, 0.6, 0.7, 0.8, 0.9, 0.99, 1 or any intermediate values or ranges derived therefrom. For a particular example, it can be set to 0.5.

The workflow 200 further includes generating similarity scores for each pair of peptides in the library based on the amino acids that make up each peptide of the pair. To accomplish this, the peptides may be aligned using any available method, such as, for example, a dynamic programming method to align sequences. For example, the Needleman Wunch algorithm or Smith-Waterman algorithm may be used.

As a particular example, a similarity of two peptides in each peptide pair may be generated by summing the similarities of the aligned pairs of peptides and normalizing by the length (len) of the peptides using Equation 2 (below) where i and j denote aligned amino acid pairs in peptides A and B. In an additional and alternate embodiment, normalization may be omitted.

$${Sim}_{peptide} ( A , B ) = \frac{{\sum }_{aligned i , j} S i {m}_{i , j}}{2 * max ( {len}_{A} , {len}_{B} ) - {\sum }_{aligned i , j} S i {m}_{i , j}} \qquad ( Equation 2 )$$

The workflow 200 groups the plurality of peptides into clusters based on the similarity scores. For example, directed Sphere Exclusion (DISE) can be used for clustering. The DISE procedure can include sorting by a property of choice, compiling a cluster seed list using a Sphere Exclusion diverse subset selection algorithm, and assigning the remaining peptides to the most similar cluster seed.

In various embodiments, the workflow 200 may include grouping the pluralities of peptides into clusters based on the similarity scores by determining a similarity threshold based on a similarity distribution. For example, a similarity distribution may be defined as a distribution of the similarity scores of each peptide in the library versus a similarity pair count, as illustrated in FIG. 4. The similarity threshold may be used to select peptides that meet or exceed the similarity threshold within each group. For example, each of the clusters includes a subset of the pluralities of peptides, and the subset of the plurality of peptides have a similarity score that are determined to meet a similarity threshold.

The similarity threshold may vary according to the chemical similarity matrix used to calculate the amino acid similarities. For example, the similarity threshold may be at least, about, or at most 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70% or any intermediate ranges or values. In particular examples, the similarity threshold may be one or more thresholds in the range of between 20 and 45%. In another example, the similarity threshold may be in the range of between 50 and 60%.

Without clustering, peptides may be sorted by replication count alone because high replication count may be an indicator for candidate binders in the multiplexed screening experiment. Clustering enriches the number of candidate binders by considering the replication count of clusters based on the quantification information of each distinct peptide in the clusters rather than individual peptides, which can provide information that particular general ‘structures’ of peptides are viable candidate binders, information that would otherwise be omitted by selecting candidate binders by distinct peptide count alone. The term “quantification information” refers to a count of copies of nucleotide or amino acid sequences. In some embodiments, the quantification information includes a count of copies of each amino acid sequence in a plurality of peptides. Each amino acid sequence can represent a distinct peptide that is different from other peptides in at least one, two, three, four, five, six, seven, eight, nine, or more amino acid positions or has a different length. After target-binding selection of a library against a desired target, the library can contain one or more instances of each distinct peptide that were selected as initial candidates for binding to the desired target.

The workflow 200 includes, at step 230, screening the clusters based on quantification information of peptides in each cluster to obtain candidates for target binding. This may be accomplished, for example, by identifying candidate clusters having quantification information over a pre-set threshold. Alternatively, this may be accomplished by ranking the clusters based on the sum total replication count of the peptides in each cluster, and selecting the top N ranked clusters (or distinct peptides, in the instance a single distinct peptide has no other cluster members) where N may vary by experiment.

The workflow 200 may further include comparing a size of each cluster and replication counts of each instance of each distinct peptide in each cluster based on the quantification information. For example, the workflow 200 may include plotting a size of each cluster and summing replication counts of each instance of each distinct peptide in each cluster based on the quantification information to identify clusters with multiple identical copies of distinct peptides. The size of each cluster is a count of the number of distinct peptides by sequence in each cluster.

The workflow 200 may further include determining a frequency of each distinct peptide in a cluster. The frequency of each distinct peptide can be determined as a replication count of instances of each distinct amino acid sequence of the peptides in the cluster based on the quantification information.

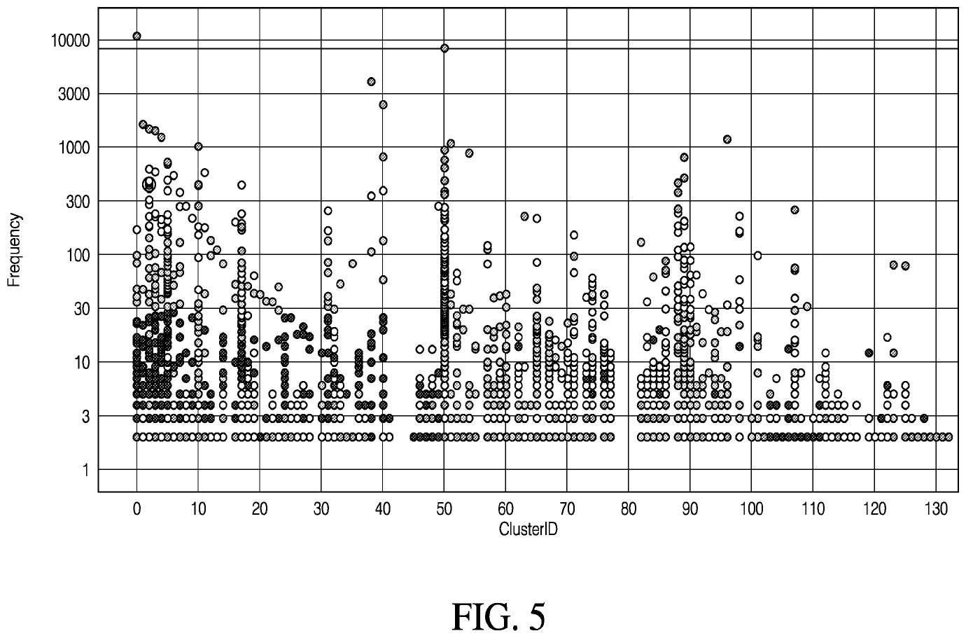

In various embodiments, the workflow 200 may further comprise visualizing clusters of peptides to screen for the candidates. For example, the workflow 200 may further comprise generating a graphic presentation to visualize a frequency of peptides in each cluster, as illustrated in FIG. 5.

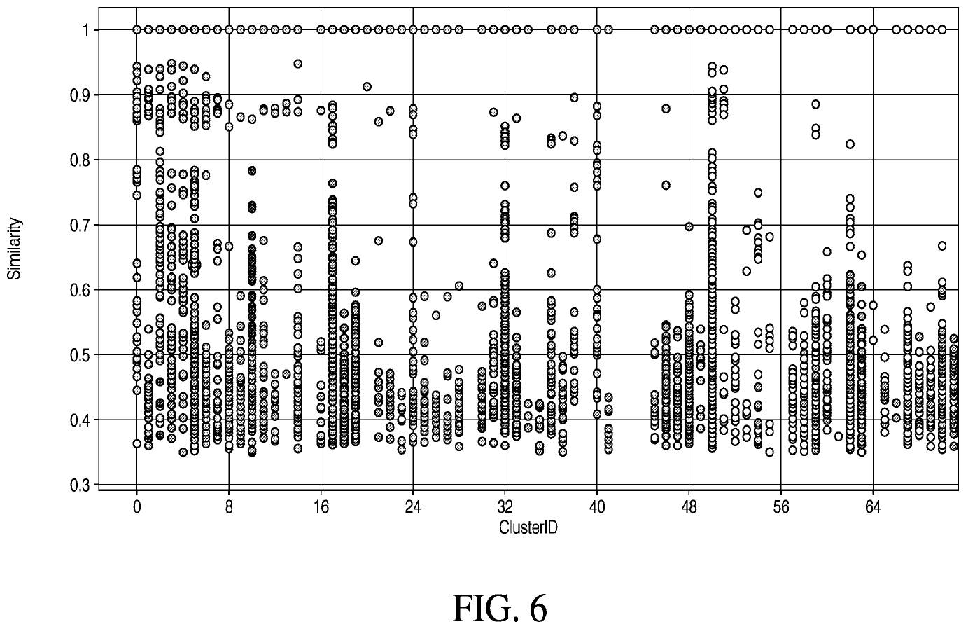

For example, the workflow 200 may further comprise generating a graphic presentation to visualize a similarity score of all peptides in each cluster, as illustrated in FIG. 6.

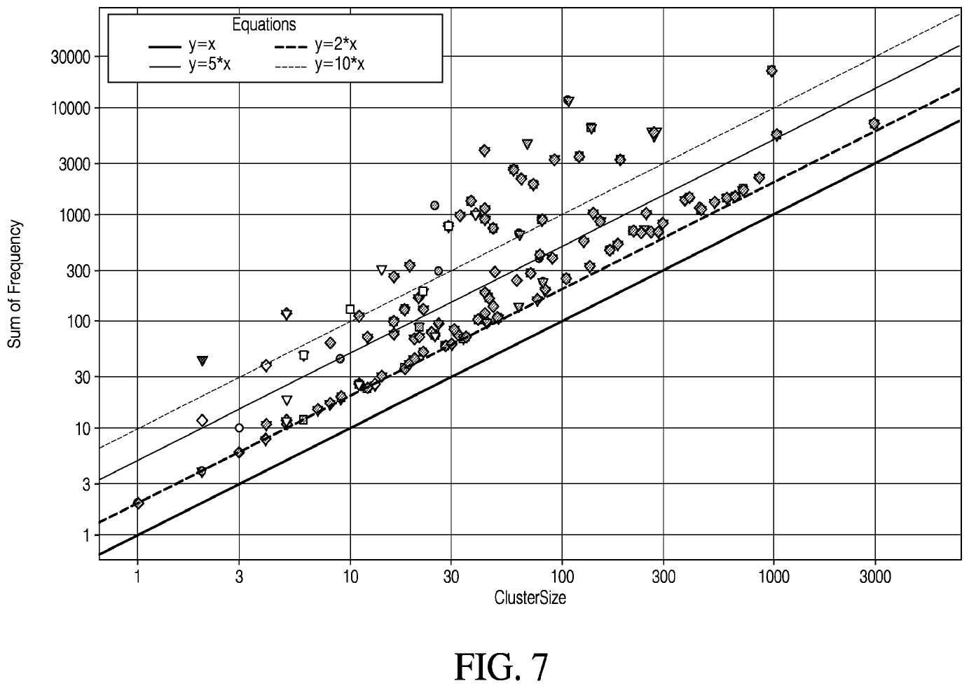

For example, the workflow 200 may further comprise generating a graphic presentation to visualize a total frequency of each cluster versus a size of each cluster, as illustrated in FIG. 7.

The workflow 200 can comprise, at step 240, validating the candidates. For example, validating the candidates may comprise preparing new peptides based on sequencing information of the candidates to test binding affinity to a desired target. For example, the workflow 200 can further comprise synthesizing the new peptides or in vitro translation of the new peptide candidates. The new peptides can be tested for binding affinity to a desired target by any binding assays or activity assays, for example, enzyme-linked immunoassay (ELISA).

IV.B. Exemplary Graphs for Clustering

FIGS. 3-7 are graphs showing non-limiting exemplary embodiments for clustering of peptides after target-binding selection.

In FIG. 3, an amino acid similarity matrix is represented. The similarity matrix has a similarity score for a comparison of each two amino acids. The similarity score can be a pre-set value between 0 and 1. For example, a comparison between D-alanine and L-alanine can generate a similarity score of 1 in a regular matrix that does not take stereochemistry difference into consideration and a similarity score of 0.109 in a stereochemistry-aware matrix. FIG. 3 represents a weighted amino acid similarity matrix that can be generated by combining a regular matrix with a first pre-determined weight (e.g., 0.5) and a stereochemistry-aware matrix with a second pre-determined weight (e.g., 0.5). For example, in the weighted amino acid similarity matrix shown in FIG. 3, a comparison between D-alanine and L-alanine can generate a similarity score of 0.6 (or, for example, 0.5545).

FIG. 4 illustrates a distribution plot of a pairwise aligned peptide (PAP) similarity score verse a similarity pair count from an exemplary library in an exemplary experiment. The x-axis represents a similarity score. This similarity score may be the pairwise aligned peptide (PAP) similarity score computed using an amino acid similarity matrix. The y-axis represents the similarity pair count, which may be a count of peptide pairs per similarity bin (i.e., per cluster). The distribution plot illustrates that a similarity threshold of 20-45% may work well for the peptide sets used in this experiment because most of the peptides have a similarity score of 20-45%. However, if a different chemical similarity is used to calculate the amino acid pair similarities, the threshold for the exactly same peptide sets could be different, such as, for example, between 50-60%. In summary, the similarity distribution of pairs of peptides is useful for selecting a similarity threshold for the clustering analysis and a means to quickly determine if the set is diverse or not.

To generate the distribution plot, up to 1 million pairs of peptides were chosen randomly from the peptides in a screening experiment. The similarity was computed for each pair of peptides. Pairs of peptides were binned in equal sized bins based on the similarities (in this case 50 bins each of size 0.02). The count of peptide pairs per similarity bin was plotted on the y axis against the minimum similarity of each bin on the x axis. Such a distribution shows how similar the peptides are to each other. The more similar peptides are to each other, the more the maximum of the distribution will move towards the right, i.e., similarity of one.

Note that the location of the maximum also depends on the chemical similarity used to generate the similarity matrix. The distribution shown in FIG. 4 is a non-limiting exemplary distribution of a diverse set of peptides using the amino acid similarity matrix generated with Atom-Atom-Path (AAP) similarity (e.g., as described in Gobbi et al., Journal of Cheminformatics (2015) 7:11, which is incorporated herein by reference in its entirety). An amino acid similarity matrix generated with ECFP (Extended Circular Fingerprint) can also be used. If the amino acid similarity matrix generated with ECFP is used, the maximum of the distribution of the same diverse set of peptides is likely to be around 0.4. The distribution of pairs of peptides is useful for selecting the threshold, i.e. the actual number, for the clustering analysis and a means to quickly determine if the set is diverse or not.

FIG. 5 illustrates a graph to show a frequency of peptides in each cluster from an exemplary library in an exemplary experiment. The y-axis shows a frequency of all peptides in each cluster, and the x-axis shows a cluster ID that identifies each cluster. Each dot represents a peptide corresponding to a frequency on the y-axis and a cluster ID on the x-axis. This illustrates that multiple clusters with a composition of peptides that may individually occur at low frequency might need further analysis after clustering based on similarity. Some peptides have low frequency individually but are clustered with similar peptides to be in a cluster with a high total frequency for all peptides assigned to the cluster. Some clusters with high frequency in aggregate relative to a pre-set threshold and their peptides may undergo further analysis.

FIG. 6 illustrates a graph to show a similarity score of all peptides in each cluster from an exemplary library in an exemplary experiment. The y-axis shows a similarity score of all peptides as compared with a corresponding cluster seed peptide in each cluster, and the x-axis shows a cluster ID for each cluster. Each dot represents a peptide corresponding to a similarity score on the y-axis and a cluster ID on the x-axis. This graph illustrates that each cluster can provide candidate peptides for further analysis based on a similarity threshold of 0.3 as exemplified here. These peptides were undergoing further analysis and were confirmed to contain several previously unidentified peptides being an inhibitor of the desired target—the inhibitors would be otherwise undetected without clustering according to the embodiments described herein.

FIG. 7 illustrates a graph to show a total frequency of all peptides in each cluster versus a size of each cluster from an exemplary library in an exemplary experiment. The y-axis shows a sum of frequencies of all peptides in each cluster, and the x-axis shows a size for each cluster, i.e., a total number of distinct peptides in each cluster (each distinct peptide may have several copies, e.g., 2, 5, 10, 100, 1000, 10,000 copies or any number or ranges derived therefrom). Each dot represents a cluster corresponding to a sum of frequencies on the y-axis and a size for each cluster on the x-axis. The lines represent y=2x, 5x, 10x for enrichment of distinct peptides in each cluster; the enrichment may be caused by directed evolution or amplification of distinct peptides during target-binding selection. For example, in a cluster on a line of y=2x, the cluster may have x=1000 distinct peptides for the cluster size, and the sum of frequency for the cluster can be 2000 that represents copies of 1000 distinct peptides all together (y=2x). This illustrates a way to identify clusters with unique peptides that would be undetected without clustering: for example, some clusters have high cluster size and low sum of frequency (close to the line representing y=x or y=2x), e. g., 1,000 or 3,000 distinct peptides but most of these peptides in these clusters don't have multiple copies so these peptides may not be detected by selection without clustering. On the other hand, some clusters may have high frequency peptides with low cluster size. These clusters may only need to select peptides with the highest frequency as the representative, but not all cluster members.

IV.C. Exemplary Clustering Methods

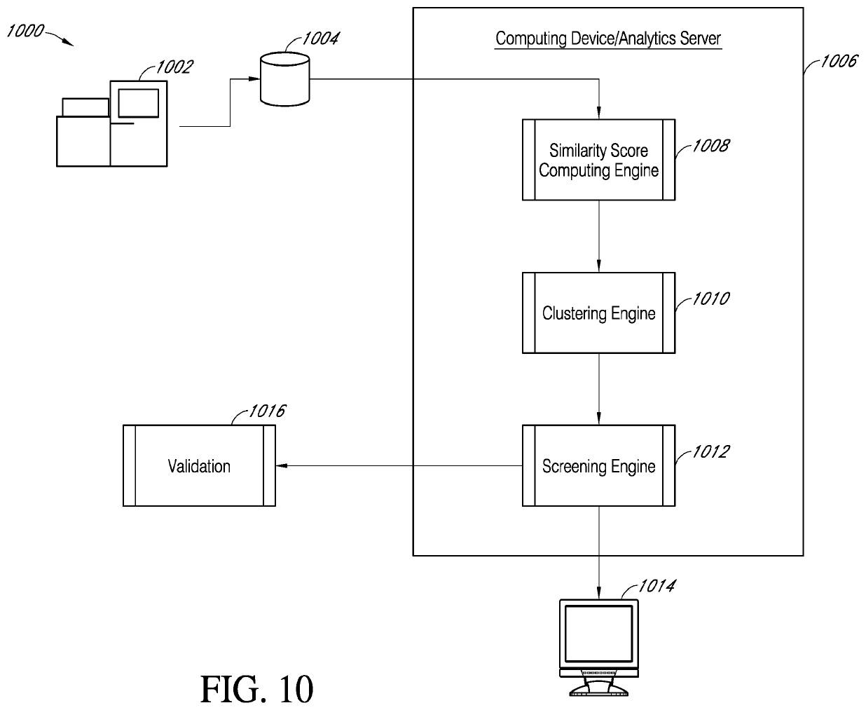

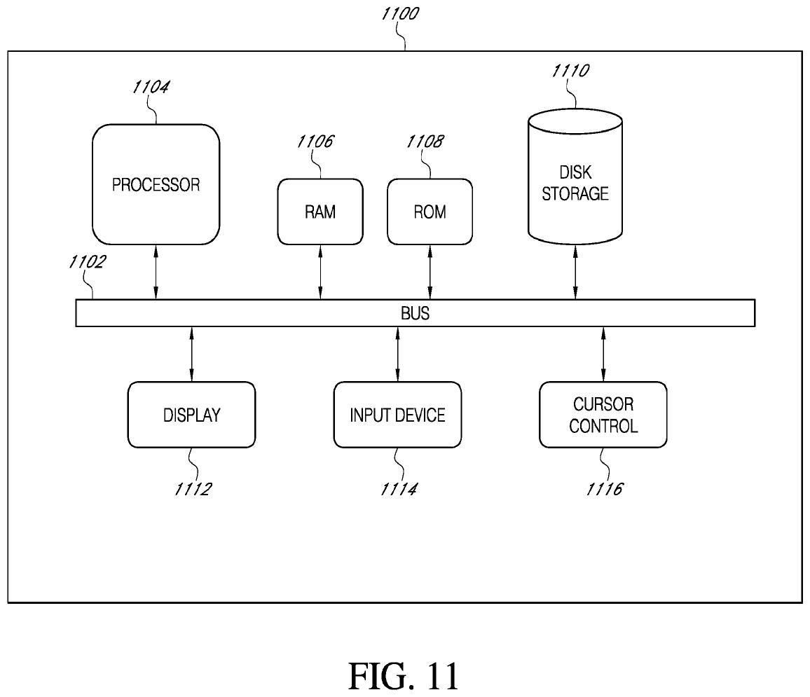

Methods are provided for detecting candidates for target binding. The methods can incorporate one or more features of the workflow 200 and can be implemented via computer software or hardware, or a combination thereof, for example, as exemplified in FIG. 10 or FIG. 11. The methods can also be implemented on a computing device/system that can include a combination of engines for detecting candidates for target binding. In various embodiments, the computing device/system can be communicatively connected to one or more of a data source, data analyzer (e.g., a clustering analyzer), and display device via a direct connection or through an internet connection.

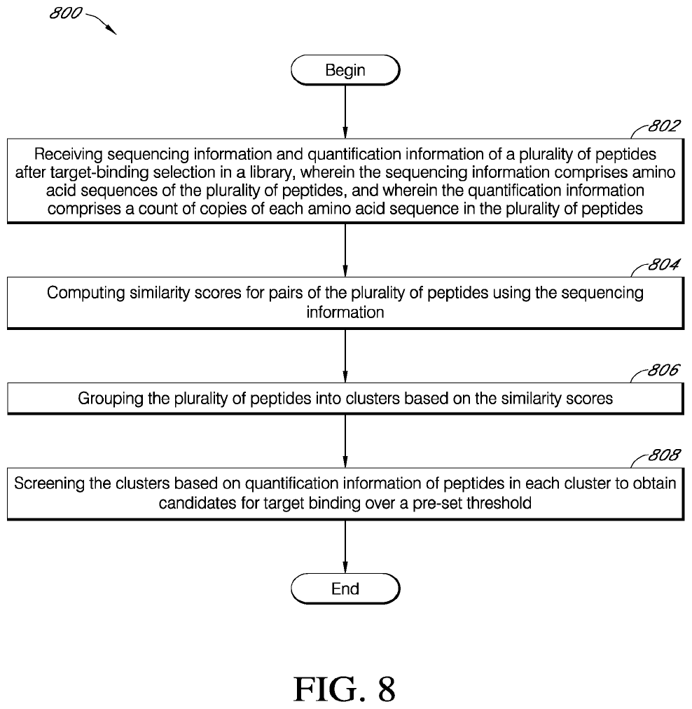

Referring now to FIG. 8, a flowchart illustrating a non-limiting example method 800 for clustering peptides to identify candidates for binding to a desired target is disclosed, in accordance with various embodiments. The method 800 can comprise, at step 802, receiving sequencing information and quantification information of a plurality of peptides after target-binding selection in a library. The sequencing information comprises amino acid sequences of the plurality of peptides in some embodiments. The quantification information comprises a count of copies of each amino acid sequence in the plurality of peptides in some embodiments. Each amino acid sequence can represent a distinct peptide that is different from other peptides in at least one, two, three, four, five, six, seven, eight, nine, or more amino acid positions or has a different length. After target-binding selection of a library against a desired target, the library can contain one or more instances of each distinct peptide that were selected as initial candidates for binding to the desired target.

The method 800 can further comprise, at step 804, computing similarity scores for pairs of the plurality of peptides using the sequencing information. For example, if a cluster seed is selected, similarity scores between any other peptide and the cluster seed in a cluster may be computed. Similarity scores between any two peptides in each cluster may also be computed in some embodiments.

In one or more embodiments, the similarity scores are computed as a numerical measure of similarity. The numerical measure of similarity for a pair of peptides may be generated based on the alignment between the two peptides. In some cases, multiple alignments for the pair of peptides may be evaluated and the alignment that provides the highest numerical measure of similarity selected. In one or more embodiments, the similarity scores are computed using an amino acid similarity matrix. The amino acid similarity matrix may include, for example, a non-stereochemistry-aware similarity matrix, a stereochemistry-aware similarity matrix, or both.

The method 800 can further comprise, at step 806, grouping the plurality of peptides into clusters based on the similarity scores. For example, grouping the plurality of peptides into clusters may comprise directed sphere exclusion clustering, conceptual clustering, hierarchical clustering, density-based spatial clustering of applications with noise (DBSCAN), or any available clustering method, or a combination thereof. In a particular example, grouping the plurality of peptides into clusters comprises directed sphere exclusion clustering. The directed sphere exclusion clustering may comprise one or more of: selecting a subset of peptides meeting a pre-determined criterion from the plurality of peptides as cluster seeds; and assigning remaining peptides in the plurality of peptides to respective cluster seeds based on the similarity scores to form clusters.

The method 800 can further comprise, at step 808, screening the clusters based on quantification information of peptides in each cluster to obtain candidates for target binding. This may be accomplished, for example, by identifying candidate clusters having quantification information over a pre-set threshold. Alternatively, this may be accomplished by ranking the clusters based on the sum total replication count of the peptides in each cluster, and selecting the top N ranked clusters (or distinct peptides, in the instance a single distinct peptide has no other cluster members) where N may vary by experiment. The top N rank may be top 1%, 5%, 10%, 20%, 30%, 40%, 50% or any intermediate ranges or values. Peptides from the candidate clusters may undergo further analysis, like binding or functional experiments to test binding activity or inhibitory functions against a desired target.

Referring now to FIG. 9, a flowchart illustrating a non-limiting example method 900 for clustering peptides to identify candidates for binding to a desired target is disclosed, in accordance with various embodiments. Method 900 may be one example of an implementation for at least a portion of the workflow 200 described above with respect to FIG. 2.

The method 900 can comprise, at step 902, receiving sequencing information for a plurality of peptides. The sequencing information may include amino acid sequences of the plurality of peptides. Each amino acid sequence can represent a distinct peptide that is different from other peptides in at least one, two, three, four, five, six, seven, eight, nine, or more amino acid positions or has a different length. After target-binding selection of a library against a desired target, the library can contain one or more instances of each distinct peptide that were selected as initial candidates for binding to the desired target.

The method 900 can comprise, at step 904, receiving quantification information for the plurality of peptides. The quantification information may include a count of copies of each amino acid sequence in the plurality of peptides. In one or more embodiments, steps 902 and 904 are performed separately. In other embodiments, steps 902 and 904 may be integrated as a single step.

The method 900 can comprise, at step 906, aligning each pair of the plurality of peptides using the sequencing information. This alignment may be performed in different ways. In one or more embodiments, a dynamic programming method may be used to align the amino acid sequences of a pair of peptides. In other embodiments, the Needleman Wunch algorithm or Smith-Waterman algorithm may be used to perform alignment.

The method 900 can comprise, at step 908, identifying an amino acid similarity matrix. Identifying the amino acid similarity matrix may include, for example, obtaining a previously generated pre-determined amino acid similarity matrix, generating an amino acid similarity matrix, or a combination of the two. The amino acid similarity matrix may be generated using, for example, a chemical similarity matrix. The chemical similarity matrix can consider the similarity in molecular structure. This type of similarity matrix enables the evaluation of unnatural amino acids. In some cases, the atom level description of the chemical similarity matrix may be used for describing differences relevant for protein-ligand interactions. For example, the chemical similarity matrix may be based on a stereochemistry-aware matrix that can distinguish amino acids based on alpha carbon (Cα) stereochemistry. The stereochemistry-aware matrix can distinguish two amino acids that are otherwise identical but have different stereo-chemistries such as, for example, different relative spatial arrangement of atoms.

In one or more embodiment, the amino acid similarity matrix identified at step 908 is generated using both a regular (non-stereochemistry-aware) amino acid similarity matrix (weighted with a first pre-determined coefficient) and a stereochemistry-aware amino acid similarity matrix (weighted with a second pre-determined coefficient). This amino acid similarity matrix provides an amino acid similarity score for each possible pairing of amino acids.

The method 900 can comprise, at step 910, computing similarity scores for the aligned pairs of the plurality of peptides using the amino acid similarity matrix. The similarity scores are computed using the amino acid similarity matrix. For example, for a given aligned pair of peptides, the amino acid similarity matrix is used to identify an amino acid similarity score for each amino acid pairing at the various positions of the aligned pair of peptides. These amino acid similarity scores are then used to compute a similarity score for the aligned pair of peptides. In one or more embodiments, the similarity score for the aligned pair of peptides is computed using the sum of the amino acid similarity scores. In some embodiments, this sum is normalized based on the lengths of the amino acid sequences of the two peptides (see, e.g., Equation 2 above). Steps 906-910 may be one example of an implementation for step 804 in FIG. 8.

The method 900 can further comprise, at step 912, grouping the plurality of peptides into clusters based on the similarity scores. For example, grouping the plurality of peptides into clusters may comprise directed sphere exclusion clustering, conceptual clustering, hierarchical clustering, density-based spatial clustering of applications with noise (DBSCAN), any other available clustering method, or a combination thereof. In a particular example, grouping the plurality of peptides into clusters comprises directed sphere exclusion clustering. The directed sphere exclusion clustering may comprise one or more of: selecting a subset of peptides meeting a pre-determined criterion from the plurality of peptides as cluster seeds. For example, prior to clustering, the plurality of peptides may be ordered by their selection experiment (in ascending order), by selection rounds (in descending order), counts (in descending order), and/or by one or more other factors. Each peptide selected as a cluster seed forms the basis for a different cluster. The remaining peptides in the plurality of peptides may be assigned to respective cluster seeds based on the similarity scores to form clusters. For example, each remaining peptide may be assigned to the cluster for which it has the highest similarity score with respect to the cluster seed. In some examples, the cluster assignments are determined based on a similarity threshold that is determined based on a distribution of the similarity scores of each peptide versus a similarity pair count.

The method 900 can further comprise, at step 914, screening the clusters based on quantification information of peptides in each cluster to obtain candidates for target binding. This may be accomplished, for example, by identifying candidate clusters having quantification information over a pre-set threshold. Alternatively, this may be accomplished by ranking the clusters based on the sum total replication count of the peptides in each cluster, and selecting the top N ranked clusters (or distinct peptides, in the instance a single distinct peptide has no other cluster members) where N may vary by experiment. The top N rank may be top 1%, 5%, 10%, 20%, 30%, 40%, 50% or any intermediate ranges or values. Peptides from the candidate clusters may undergo further analysis, like binding or functional experiments to test binding activity or inhibitory functions against a desired target.

IV.D. Exemplary Clustering Systems

In various embodiments, any methods for clustering similar peptides after target-binding selection or as exemplified in workflow 200, method 800, and/or method 900 can be implemented via software, hardware, firmware, or a combination thereof, such as described in FIG. 10. FIG. 10 illustrates a non-limiting example system configured to clustering similar peptides in target-binding selection, in accordance with various embodiments. The system 1000 can include various combinations of features, whether it be more or less features than that are illustrated in FIG. 10. As such, FIG. 10 simply illustrates one example of a possible system.

The system 1000 includes a data collection unit 1002, a data storage unit 1004, a computing device/analytics server 1006, a display 1014, and a validation unit 1016. The data collection unit 1002 may be a sequencing instrument, a quantification instrument such as quantitative PCR instrument, or a combination thereof. A sequencing instrument obtains sequencing information of DNA components in peptide conjugates after target-binding selection. The sequencing instrument can be a next generation sequencing instrument. A quantitative PCR instrument is a machine that amplifies and detects DNA and combines the functions of a thermal cycler and a fluorimeter, enabling the process of quantitative PCR. Quantitative PCR instruments monitor the progress of PCR, and the nature of amplified products, by measuring fluorescence. The data collection unit 1002 can also obtain sequencing information and quantification information of peptides in the peptide-DNA conjugates based on the sequences and quantities of DNA components in the peptide-DNA conjugates.

The data collection unit 1002 can be communicatively connected to and can send datasets to the data storage unit 1004 by way of a serial bus (if both form an integrated instrument platform) or by way of a network connection (if both are distributed/separate devices). The generated datasets are stored in the data storage unit 1004 for subsequent processing. In various embodiments, one or more raw datasets can also be stored in the data storage unit 1004 prior to processing and analyzing. Accordingly, in various embodiments, the data storage unit 604 can be configured to store datasets of the various embodiments herein that correspond to a plurality of libraries of DNA-peptide conjugates. In various embodiments, the processed and analyzed datasets can be fed to the computing device/analytics server 1006 in real-time for further downstream analysis.

The data storage unit 1004 can be communicatively connected to the computing device/analytics server 1006. In various embodiments, the data storage unit 1004 and the computing device/analytics server 1006 can be part of an integrated apparatus. In various embodiments, the data storage unit 1004 can be hosted by a different device than the computing device/analytics server 1006. In various embodiments, the data storage unit 1004 and the computing device/analytics server 1006 can be part of a distributed network system. In various embodiments, the computing device/analytics server 1006 can be communicatively connected to the data storage unit 604 via a network connection that can be either a “hardwired” physical network connection (e.g., Internet, LAN, WAN, VPN, etc.) or a wireless network connection (e.g., Wi-Fi, WLAN, etc.). The computing device/analytics server 1006 can be a workstation, mainframe computer, distributed computing node (part of a “cloud computing” or distributed networking system), personal computer, mobile device, etc, according to various embodiments. The computing device/analytics server 1006 can be a client computing device. In various embodiments, the computing device/analytics server 1006 can be a personal computing device having a web browser (e.g., INTERNET EXPLORER™, FIREFOX™ SAFARI™ etc.) that can be used to control the operation of the data collection unit 1002, data storage unit 1004, display 1014, and validation unit 1016.

The computing system such as computer device/analytics sever 1006 is configured to host one or more similarity score computing engines 1008, one or more clustering engines 1010, and one or more screening engines 1012, according to various embodiments. The similarity score computing engine 1008 is configured to obtain or receive sequencing information and quantification information of a plurality of peptides after target-binding selection in a library and compute similarity scores for pairs of the plurality of peptides using the sequencing information. In various embodiments, the sequencing information comprises amino acid sequences of the plurality of peptides, and the quantification information comprises a count of copies of each amino acid sequence in the plurality of peptides. The clustering engine 1010 is configured to group the plurality of peptides into clusters based on the similarity scores. The screening engine 1012 is configured to screen the clusters based on quantification information of peptides in each cluster to obtain candidates for target binding over a pre-set threshold.

The system 1000 further comprises a validation unit 1016 configured to validate selected candidates from the libraries based on the screening results.

During the time when the computing device/analytics server 1006 is receiving and processing data from the data storage unit 1004 or after the processing is done, an output of the results can be displayed as a result or summary on a display 1014 that is communicatively connected to the computing device/analytics server 1006. The display 1014 can be a client computing device or a client terminal. The display 1014 can be a personal computing device having a web browser (e.g., INTERNET EXPLORER™, FIREFOX™, SAFARI™, etc.) that can be used to control the operation of the operation of the data collection unit 1002, data storage unit 1004, similarity score computing engine 1008, clustering engines 1010, screening engine 1012, and display 1014.

It should be appreciated that the various engines can be combined or collapsed into a single engine, component or module, depending on the requirements of the particular application or system architecture. Engines 1008/1010/1012 can comprise additional engines or components as needed by the particular application or system architecture.

### V. Computer-Implemented System

In various embodiments, any methods for clustering similar peptides after target-binding selection or as exemplified in workflow 200, method 800, and/or method 900 can be implemented via software, hardware, firmware, or a combination thereof, such as described in FIG. 10 or FIG. 11.

That is, as depicted in FIG. 10, the methods disclosed herein can be implemented on a computer system such as computer system 1006 (e.g., a computing device/analytics server). The computer system 1006 (e.g., a computing device/analytics server) can be communicatively connected to a data storage 1004 and a display system 1014 via a direct connection or through a network connection (e.g., LAN, WAN, Internet, etc.). It should be appreciated that the computer system 1006 (e.g., a computing device/analytics server) depicted in FIG. 10 can comprise additional engines or components as needed by the particular application or system architecture.

FIG. 11 is a block diagram illustrating a computer system 1100 upon which embodiments of the present teachings may be implemented. In various embodiments of the present teachings, computer system 1100 can include a bus 1102 or other communication mechanism for communicating information and a processor 1104 coupled with bus 1102 for processing information. In various embodiments, computer system 1100 can also include a memory, which can be a random-access memory (RAM) 1106 or other dynamic storage device, coupled to bus 1102 for determining instructions to be executed by processor 1104. Memory can also be used for storing temporary variables or other intermediate information during execution of instructions to be executed by processor 1104. In various embodiments, computer system 1100 can further include a read only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, processor 1104 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communication of information and command selections to processor 1104. Another type of user input device is a cursor control, such as a mouse, a trackball or cursor direction keys for communicating direction information and command selections to processor 1104 and for controlling cursor movement on display 1112.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, dynamic memory, such as memory 1106. Examples of transmission media can include, but are not limited to, coaxial cables, copper wire, and fiber optics, including the wires that comprise bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 900 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, memory components 1106/1108/1110 and user input provided via input device 1114.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### VI. Recitation of Embodiments

Embodiment 1. A method for detecting candidates for target binding, the method comprising: receiving sequencing information and quantification information of a plurality of peptides after target-binding selection in a library, wherein the sequencing information comprises amino acid sequences of the plurality of peptides, and wherein the quantification information comprises a count of copies of each amino acid sequence in the plurality of peptides; computing similarity scores for pairs of the plurality of peptides using the sequencing information; grouping the plurality of peptides into clusters based on the similarity scores; and screening the clusters based on quantification information of peptides in each cluster to obtain candidates for target binding over a pre-set threshold.

Embodiment 2. The method of embodiment 1, further comprising: aligning each pair of the plurality of peptides using the sequencing information to generate a numerical measure of similarity for each pair of the plurality of peptides.

Embodiment 3. The method of embodiment 2, wherein computing the similarity scores between any pair of peptides comprises using a numerical measure of similarity based on an alignment between peptides of each pair.

Embodiment 4. The method of any one of embodiments 1-3, further comprising: computing the similarity scores for each of the pairs using an amino acid similarity matrix.

Embodiment 5. The method of embodiment 4, further comprising: obtaining or generating the amino acid similarity matrix.

Embodiment 6. The method of embodiment 4 or embodiment 5, wherein the amino acid similarity matrix comprises a chemical similarity matrix.

Embodiment 7. The method of embodiment 6, wherein the chemical similarity matrix distinguishes amino acids based on alpha carbon (Cα) stereochemistry.

Embodiment 8. The method of any one of embodiments 4-5, wherein the amino acid similarity matrix comprises a combination of a regular amino acid similarity matrix via a first pre-determined coefficient and a stereochemistry-aware amino acid similarity matrix via a second pre-determined coefficient.

Embodiment 9. The method of any one of embodiments 1-8, wherein computing the similarity scores for the pairs of the plurality of peptides comprises normalizing based on lengths of peptides for each of the pairs of the plurality of peptides.

Embodiment 10. The method of any one of embodiments 1-9, wherein grouping the plurality of peptides into clusters comprises directed sphere exclusion clustering, conceptual clustering, hierarchical clustering, density-based spatial clustering of applications with noise (DBSCAN), or a combination thereof.

Embodiment 11. The method of embodiment any one of embodiments 1-10, wherein grouping the plurality of peptides into clusters comprises: selecting a subset of peptides meeting a pre-determined criterion from the plurality of peptides as cluster seeds; and assigning remaining peptides in the plurality of peptides to respective cluster seeds based on the similarity scores to form clusters.

Embodiment 12. The method of any one of embodiments 1-11, wherein grouping the pluralities of peptides into clusters based on the similarity scores comprises determining a similarity threshold based on a similarity distribution that is defined as a distribution of the similarity scores of each peptide in the library versus a similarity pair count.

Embodiment 13. The method of embodiment 12, wherein the similarity threshold is a similarity between 20-45%.

Embodiment 14. The method of any one of embodiments 1-13, wherein each of the clusters comprises a subset of the plurality of peptides, and wherein each peptide in the subset of the plurality of peptides paired with a cluster seed of the cluster has a similarity score that is determined to meet a similarity threshold.

Embodiment 15. The method of any one of embodiments 1-14, further comprising ranking the clusters by summing replication counts of each instance of each distinct peptide in each cluster based on the quantification information.

Embodiment 16. The method of any one of embodiments 1-15, further comprising correlating a size of each cluster with a sum of replication counts of all instances of each distinct peptide in each cluster based on the quantification information to identify peptides based on the correlation, wherein the size of each cluster is a count of distinct peptides by sequence in each cluster.

Embodiment 17. The method of any one of embodiments 1-16, wherein the peptides comprise DNA-RNA-macrocycle conjugates.

Embodiment 18. The method of any one of embodiments 1-17, wherein at least one of the peptides comprises natural and non-natural amino acids.

Embodiment 19. The method of any one of embodiments 1-18, wherein the plurality of peptides are made using a codon table encoding natural amino acids, a codon table encoding non-natural amino acids, or a combination thereof.

Embodiment 20. The method of embodiment 17, wherein the quantification information of the plurality of peptides is determined from counting DNA copies in the DNA-RNA-macrocycle conjugates.

Embodiment 21. The method of any one of embodiments 17-20, wherein the sequencing information of the plurality of peptides is determined from corresponding DNA sequences in the DNA-RNA-macrocycle conjugates.

Embodiment 22. The method of any one of embodiments 1-21, wherein the target-binding selection comprises: performing in vitro transcription of a DNA library to produce mRNA; performing in vitro translation on mRNA to produce RNA-peptide conjugates; performing in vitro reverse transcription on the RNA-peptide conjugates to produce input DNA-RNA-peptides; incubating the input DNA-RNA-peptides with a desired target; and selecting for target-binding DNA-RNA-peptides from the input DNA-RNA-peptides, wherein the target-binding DNA-RNA-peptides are initial candidates that bind the desired target and are defined as the plurality of peptides after the target-binding selection.

Embodiment 23. The method of embodiment 22, further comprising amplifying the target-binding DNA-RNA-peptides by PCR to determine the quantification information for the plurality of peptides after target-binding selection.

Embodiment 24. The method of embodiment 22 or embodiment 23, further comprising sequencing the target-binding DNA-RNA-peptides to determine the sequencing information for the plurality of peptides after target-binding selection.

Embodiment 25. The method of any one of embodiments 1-24, further comprising validating the candidates by preparing new peptide candidates based on sequence information of the candidates to test binding affinity to a desired target.

Embodiment 26. The method of embodiment 25, further comprising synthetizing the new peptide candidates.

Embodiment 27. The method of embodiment 25 or embodiment 26, further comprising in vitro translation of the new peptide candidates.

Embodiment 28. The method of any one of embodiments 1-27, further comprising determining a frequency of each sequence in a cluster as a replication count of each instance of a distinct peptide in the cluster based on the quantification information.

Embodiment 29. The method of embodiment 28, further comprising generating a graphic presentation to visualize a sum of frequencies of amino acid sequences in each cluster.

Embodiment 30. The method of embodiment 28 or embodiment 29, further comprising generating a graphic presentation to visualize a sum of frequencies of amino acid sequences in each cluster versus a size of each cluster.

Embodiment 31. The method of any one of embodiments 1-30, further comprising generating a graphic presentation to visualize a similarity score of all peptides in each cluster.

Embodiment 32. A computer-program product tangibly embodied in a non-transitory machine-readable storage medium, including instructions configured to cause one or more data processors to perform a method for detecting candidates for target binding, the method comprising: receiving sequencing information and quantification information of a plurality of peptides after target-binding selection in a library, wherein the sequencing information comprises amino acid sequences of the plurality of peptides, and wherein the quantification information comprises a count of copies of each amino acid sequence in the plurality of peptides; computing similarity scores for pairs of the plurality of peptides using the sequencing information; grouping the plurality of peptides into clusters based on the similarity scores; and screening the clusters based on quantification information of peptides in each cluster to obtain candidates for target binding over a pre-set threshold.

Embodiment 33. The computer-program product of embodiment 32, wherein the method further comprises aligning each pair of the plurality of peptides using the sequencing information to generate a numerical measure of similarity for each pair.

Embodiment 34. The computer-program product of embodiment 33, wherein computing the similarity scores between any pair of peptides comprises using a numerical measure of similarity based on an alignment between peptides of each pair.

Embodiment 35. The computer-program product of any one of embodiments 32-34, wherein the method further comprises computing the similarity scores for each of the pairs using an amino acid similarity matrix.

Embodiment 36. The computer-program product of embodiment 35, wherein the method further comprises obtaining or generating the amino acid similarity matrix.

Embodiment 37. The computer-program product of embodiment 35 or embodiment 36, wherein the amino acid similarity matrix comprises a chemical similarity matrix.

Embodiment 38. The computer-program product of embodiment 37, wherein the chemical similarity matrix distinguishes amino acids based on alpha carbon (Cα) stereochemistry.

Embodiment 39. The computer-program product of any one of embodiments 35-37, wherein the amino acid similarity matrix comprises a combination of a regular amino acid similarity matrix via a first pre-determined coefficient and a stereochemistry-aware amino acid similarity matrix via a second pre-determined coefficient.

Embodiment 40. The computer-program product of any one of embodiments 32-39, wherein computing the similarity scores for the pairs of the plurality of peptides comprises normalizing based on lengths of peptides for each of the pairs of the plurality of peptides.

Embodiment 41. The computer-program product of any one of embodiments 32-40, wherein grouping the plurality of peptides into clusters comprises directed sphere exclusion clustering, conceptual clustering, hierarchical clustering, density-based spatial clustering of applications with noise (DBSCAN), or a combination thereof.

Embodiment 42. The computer-program product of any one of embodiments 32-41, wherein grouping the plurality of peptides into clusters comprises: selecting a subset of peptides meeting a pre-determined criterion from the plurality of peptides as cluster seeds; and assigning remaining peptides in the plurality of peptides to respective cluster seeds based on the similarity scores to form clusters.

Embodiment 43. The computer-program product of any one of embodiments 32-42, wherein grouping the pluralities of peptides into clusters based on the similarity scores comprises determining a similarity threshold based on a similarity distribution that is defined as a distribution of the similarity scores of each peptide in the library versus a similarity pair count.

Embodiment 44. The computer-program product of embodiment 43, wherein the similarity threshold is a similarity between 20-45%.

Embodiment 45. The computer-program product of any one of embodiments 32-44, wherein each of the clusters comprises a subset of the plurality of peptides, and wherein each peptide in the subset of the plurality of peptides paired with a cluster seed of the cluster has a similarity score that is determined to meet a similarity threshold.

Embodiment 46. The computer-program product of any one of embodiments 32-45, wherein the method further comprises ranking the clusters by summing replication counts of each instance of each distinct peptide in each cluster based on the quantification information.

Embodiment 47. The computer-program product of any one of embodiments 32-46, wherein the method further comprises correlating a size of each cluster and with a sum of replication counts of all instances of each distinct peptide in each cluster based on the quantification information to identify peptides based on the correlation, wherein the size of each cluster is a count of distinct peptides by sequence in each cluster.

Embodiment 48. The computer-program product of any one of embodiments 32-47, wherein the peptides comprise DNA-RNA-macrocycle conjugates.

Embodiment 49. The computer-program product of embodiment 48, wherein the quantification information of the plurality of peptides is determined from counting DNA copies in the DNA-RNA-macrocycle conjugates.

Embodiment 50. The computer-program product of embodiment 48 or embodiment 49, wherein the sequencing information of the plurality of peptides is determined from corresponding DNA sequences in the DNA-RNA-macrocycle conjugates.

Embodiment 51. The computer-program product of any one of embodiments 32-50, wherein the method further comprises determining a frequency of each sequence in a cluster as a replication count of each instance of a distinct peptide in the cluster based on the quantification information.

Embodiment 52. The computer-program product of embodiment 51, wherein the method further comprises generating a graphic presentation to visualize a sum of frequencies of amino acid sequences in each cluster.

Embodiment 53. The computer-program product of embodiment 51 or embodiment 52, wherein the method further comprises generating a graphic presentation to visualize a sum of frequencies of amino acid sequences in each cluster versus a size of each cluster.

Embodiment 54. The computer-program product of any one of embodiments 32-50, wherein the method further comprises generating a graphic presentation to visualize a similarity score of all peptides in each cluster.

Embodiment 55. A system comprising: a data store configured to store a dataset containing sequencing information and quantification information of a plurality of peptides after target-binding selection in a library, wherein the sequencing information comprises amino acid sequences of the plurality of peptides, and wherein the quantification information comprises a count of copies of each amino acid sequence in the plurality of peptides; one or more data processors; and a computing device communicatively connected to the data store and configured to receive the data set, the computing device comprising a non-transitory computer readable storage medium containing instructions which, when executed on the one or more data processors, cause the one or more data processors to perform a method for detecting candidates for target binding, the method comprising: computing similarity scores for pairs of the plurality of peptides using the sequencing information; grouping the plurality of peptides into clusters based on the similarity scores; and screening the clusters based on quantification information of peptides in each cluster to obtain candidates for target binding over a pre-set threshold.

Embodiment 56. The system of embodiment 55, wherein the method further comprises aligning each pair of the plurality of peptides using the sequencing information to generate a numerical measure of similarity for each pair of the plurality of peptides.

Embodiment 57. The system of embodiment 56, wherein computing the similarity scores between any pair of peptides comprises using a numerical measure of similarity based on an alignment between peptides of each pair.

Embodiment 58. The system of any one of embodiments 55-57, wherein the method further computing the similarity scores for each of the pairs using an amino acid similarity matrix.

Embodiment 59. The system of embodiment 58, wherein the method further comprises generating the amino acid similarity matrix.

Embodiment 60. The system of embodiment 58 or embodiment 59, wherein the amino acid similarity matrix comprises a chemical similarity matrix.

Embodiment 61. The system of embodiment 60, wherein the chemical similarity matrix distinguishes amino acids based on alpha carbon (Cα) stereochemistry.

Embodiment 62. The system of any one of embodiments 55-60, wherein the amino acid similarity matrix comprises a combination of a regular amino acid similarity matrix via a first pre-determined coefficient and a stereochemistry-aware amino acid similarity matrix via a second pre-determined coefficient.

Embodiment 63. The system of any one of embodiments 55-63, wherein computing the similarity scores for the pairs of the plurality of peptides comprises normalizing based on lengths of peptides for each of the pairs of the plurality of peptides.

Embodiment 64. The system of any one of embodiments 55-63, wherein grouping the plurality of peptides into clusters comprises directed sphere exclusion clustering, conceptual clustering, hierarchical clustering, density-based spatial clustering of applications with noise (DBSCAN), or a combination thereof.

Embodiment 65. The system of any one of embodiments 55-64, wherein grouping the plurality of peptides into clusters comprises: selecting a subset of peptides meeting a pre-determined criterion from the plurality of peptides as cluster seeds; and assigning remaining peptides in the plurality of peptides to respective cluster seeds based on the similarity scores to form clusters.

Embodiment 66. The system of any one of embodiments 55-57, wherein grouping the pluralities of peptides into clusters based on the similarity scores comprises determining a similarity threshold based on a similarity distribution that is defined as a distribution of the similarity scores of each peptide in the library versus a similarity pair count.

Embodiment 67. The system of embodiment 66, wherein the similarity threshold is a similarity between 20-45%.

Embodiment 68. The system of any one of embodiments 55-67, wherein each of the clusters comprises a subset of the plurality of peptides, and wherein each peptide in the subset of the plurality of peptides paired with a cluster seed of the cluster has a similarity score that is determined to meet a similarity threshold.

Embodiment 69. The system of any one of embodiments 55-68, further comprising ranking the clusters by summing replication counts of each instance of each distinct peptide in each cluster based on the quantification information.

Embodiment 70. The system of any one of embodiments 55-69, wherein the method further comprises correlating a size of each cluster with a sum of replication counts of all instances of each distinct peptide in each cluster based on the quantification information to identify peptides based on the correlation, wherein the size of each cluster is a count of distinct peptides by sequence in each cluster.

Embodiment 71. The system of any one of embodiments 55-70, wherein the peptides comprise DNA-RNA-macrocycle conjugates.

Embodiment 72. The system of embodiment 71, wherein the quantification information of the plurality of peptides is determined from counting DNA copies in the DNA-RNA-macrocycle conjugates.

Embodiment 73. The system of embodiment 71 or embodiment 72, wherein the sequencing information of the plurality of peptides is determined from corresponding DNA sequences in the DNA-RNA-macrocycle conjugates.

Embodiment 74. The system of any one of embodiments 55-73, wherein the method further comprises determining a frequency of each sequence in a cluster as a replication count of each instance of a distinct peptide in the cluster based on the quantification information.

Embodiment 75. The system of embodiment 74, wherein the method further comprises generating a graphic presentation to visualize a sum of frequencies of amino acid sequences in each cluster.

Embodiment 76. The system of embodiment 74 or embodiment 75, wherein the method further comprises generating a graphic presentation to visualize a sum of frequencies of amino acid sequences in each cluster versus a size of each cluster.

Embodiment 77. The system of any one of embodiments 55-76, wherein the method further comprises generating a graphic presentation to visualize a similarity score of all peptides in each cluster.

### VII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

All references cited herein, including patent applications, patent publications, and UniProtKB/Swiss-Prot Accession numbers are herein incorporated by reference in their entirety, as if each individual reference were specifically and individually indicated to be incorporated by reference.

What is claimed is:

1. A method for detecting candidates for target binding, the method comprising:
receiving sequencing information and quantification information of a plurality of peptides after target-binding selection in a library,
wherein the sequencing information comprises amino acid sequences of the plurality of peptides, and
wherein the quantification information comprises a count of copies of each amino acid sequence in the plurality of peptides;
computing similarity scores for pairs of the plurality of peptides using the sequencing information;
grouping the plurality of peptides into clusters based on the similarity scores; and
screening the clusters based on quantification information of peptides in each cluster to obtain candidates for target binding over a pre-set threshold.

2. The method of claim 1, further comprising:
aligning each pair of the plurality of peptides using the sequencing information to generate a numerical measure of similarity for each pair of the plurality of peptides.

3. The method of claim 2, wherein computing the similarity scores between any pair of peptides comprises using a numerical measure of similarity based on an alignment between peptides of each pair.

4. The method of claim 1, further comprising:
computing the similarity scores for each of the pairs using an amino acid similarity matrix.

5. The method of claim 4, further comprising:
obtaining or generating the amino acid similarity matrix.

6. The method of claim 4, wherein the amino acid similarity matrix comprises a chemical similarity matrix.

7. The method of claim 6, wherein the chemical similarity matrix distinguishes amino acids based on alpha carbon (Cα) stereochemistry.

8. The method of claim 4, wherein the amino acid similarity matrix comprises a combination of a regular amino acid similarity matrix via a first pre-determined coefficient and a stereochemistry-aware amino acid similarity matrix via a second pre-determined coefficient.

9. The method of claim 1, wherein computing the similarity scores for the pairs of the plurality of peptides comprises normalizing based on lengths of peptides for each of the pairs of the plurality of peptides.

10. The method of claim 1, wherein grouping the plurality of peptides into clusters comprises directed sphere exclusion clustering, conceptual clustering, hierarchical clustering, density-based spatial clustering of applications with noise (DBSCAN), or a combination thereof.

11. The method of claim 1, wherein grouping the plurality of peptides into clusters comprises:
selecting a subset of peptides meeting a pre-determined criterion from the plurality of peptides as cluster seeds; and
assigning remaining peptides in the plurality of peptides to respective cluster seeds based on the similarity scores to form clusters.

12. The method of claim 1, wherein grouping the pluralities of peptides into clusters based on the similarity scores comprises determining a similarity threshold based on a similarity distribution that is defined as a distribution of the similarity scores of each peptide in the library versus a similarity pair count.

13. The method of claim 12, wherein the similarity threshold is a similarity between 20-45%.

14. The method of claim 1, wherein each of the clusters comprises a subset of the plurality of peptides, and wherein each peptide in the subset of the plurality of peptides paired with a cluster seed of the cluster has a similarity score that is determined to meet a similarity threshold.

15. The method of claim 1, further comprising ranking the clusters by summing replication counts of each instance of each distinct peptide in each cluster based on the quantification information.

16. The method of claim 1, further comprising correlating a size of each cluster with a sum of replication counts of all instances of each distinct peptide in each cluster based on the quantification information to identify peptides based on the correlation, wherein the size of each cluster is a count of distinct peptides by sequence in each cluster.

17. A computer-program product tangibly embodied in a non-transitory machine-readable storage medium, including instructions configured to cause one or more data processors to perform a method for detecting candidates for target binding, the method comprising:
receiving sequencing information and quantification information of a plurality of peptides after target-binding selection in a library, wherein the sequencing information comprises amino acid sequences of the plurality of peptides, and wherein the quantification information comprises a count of copies of each amino acid sequence in the plurality of peptides;
computing similarity scores for pairs of the plurality of peptides using the sequencing information;
grouping the plurality of peptides into clusters based on the similarity scores; and
screening the clusters based on quantification information of peptides in each cluster to obtain candidates for target binding over a pre-set threshold.

18. A system comprising:
a data store configured to store a dataset containing sequencing information and quantification information of a plurality of peptides after target-binding selection in a library, wherein the sequencing information comprises amino acid sequences of the plurality of peptides, and wherein the quantification information comprises a count of copies of each amino acid sequence in the plurality of peptides;
one or more data processors; and
a computing device communicatively connected to the data store and configured to receive the data set, the computing device comprising a non-transitory computer readable storage medium containing instructions which, when executed on the one or more data processors, cause the one or more data processors to perform a method for detecting candidates for target binding, the method comprising:
computing similarity scores for pairs of the plurality of peptides using the sequencing information;
grouping the plurality of peptides into clusters based on the similarity scores; and
screening the clusters based on quantification information of peptides in each cluster to obtain candidates for target binding over a pre-set threshold.

19. The system of claim 18, wherein the method further comprises aligning each pair of the plurality of peptides using the sequencing information to generate a numerical measure of similarity for each pair of the plurality of peptides.

20. The system of claim 19, wherein computing the similarity scores between any pair of peptides comprises using a numerical measure of similarity based on an alignment between peptides of each pair.

21. The system of claim 18, wherein the method further computing the similarity scores for each of the pairs using an amino acid similarity matrix.

22. The system of claim 21, wherein the method further comprises generating the amino acid similarity matrix.

23. The system of claim 21, wherein the amino acid similarity matrix comprises a chemical similarity matrix.

24. The system of claim 23, wherein the chemical similarity matrix distinguishes amino acids based on alpha carbon (Cα) stereochemistry.

25. The system of claim 21, wherein the amino acid similarity matrix comprises a combination of a regular amino acid similarity matrix via a first pre-determined coefficient and a stereochemistry-aware amino acid similarity matrix via a second pre-determined coefficient.

26. The system of claim 18, wherein computing the similarity scores for the pairs of the plurality of peptides comprises normalizing based on lengths of peptides for each of the pairs of the plurality of peptides.

27. The system of claim 18, wherein grouping the plurality of peptides into clusters comprises directed sphere exclusion clustering, conceptual clustering, hierarchical clustering, density-based spatial clustering of applications with noise (DBSCAN), or a combination thereof.

28. The system of claim 18, wherein grouping the plurality of peptides into clusters comprises:
selecting a subset of peptides meeting a pre-determined criterion from the plurality of peptides as cluster seeds; and
assigning remaining peptides in the plurality of peptides to respective cluster seeds based on the similarity scores to form clusters.

29. The system of claim 18, wherein grouping the pluralities of peptides into clusters based on the similarity scores comprises determining a similarity threshold based on a similarity distribution that is defined as a distribution of the similarity scores of each peptide in the library versus a similarity pair count.

30. The system of claim 29, wherein the similarity threshold is a similarity between 20-45%.

31. The system of claim 18, wherein each of the clusters comprises a subset of the plurality of peptides, and wherein each peptide in the subset of the plurality of peptides paired with a cluster seed of the cluster has a similarity score that is determined to meet a similarity threshold.

32. The system of claim 18, further comprising ranking the clusters by summing replication counts of each instance of each distinct peptide in each cluster based on the quantification information.

33. The system of claim 18, wherein the method further comprises correlating a size of each cluster with a sum of replication counts of all instances of each distinct peptide in each cluster based on the quantification information to identify peptides based on the correlation, wherein the size of each cluster is a count of distinct peptides by sequence in each cluster.
